SEQUENCE LISTING (28 sequences)

SEQ ID NO: 1 - protein, 5 aa, Homo sapiens
  MISC_FEATURE: GPC3.1 heavy chain CDR1
SYWIA

SEQ ID NO: 2 - protein, 17 aa, Homo sapiens
  MISC_FEATURE: GPC3.1 heavy chain CDR2
IIFPGDSDTRYSPSFQG

SEQ ID NO: 3 - protein, 8 aa, Homo sapiens
  MISC_FEATURE: GPC3.1 heavy chain CDR3
TREGYFDY

SEQ ID NO: 4 - protein, 12 aa, Homo sapiens
  MISC_FEATURE: GPC3.1 kappa chain CDR1
RAVQSVSSSYLA

SEQ ID NO: 5 - protein, 7 aa, Homo sapiens
  MISC_FEATURE: GPC3.1 kappa chain CDR2
GASSRAT

SEQ ID NO: 6 - protein, 8 aa, Homo sapiens
  MISC_FEATURE: GPC3.1 kappa chain CDR3
QQYGSSPT

SEQ ID NO: 7 - protein, 117 aa, Homo sapiens
  MISC_FEATURE: GPC3.1 heavy chain variable region
  MISC_FEATURE (31)..(35): GPC3.1 VH CDR1
  MISC_FEATURE (50)..(66): GPC3.1 VH CDR2
  MISC_FEATURE (99)..(106): GPC3.1 VH CDR3
EVQLVQSGAEVKKPGESLKISCKGSGYSFTSYWIAWVRQMPGKGLEWMGIIFPGDSDTRYSPSFQGQVTISADRSIRTAYLQWSSLKASDTALYYCARTREGYFDYWGQGTLVTVSS

SEQ ID NO: 8 - protein, 107 aa, Homo sapiens
  MISC_FEATURE: GPC3.1 kappa chain variable region
  MISC_FEATURE (24)..(34): GPC3.1 VK CDR1
  MISC_FEATURE (51)..(57): GPC3.1 VK CDR2
  MISC_FEATURE (90)..(97): GPC3.1 VK CDR3
EIVLTQSPGTLSLSPGERATLSCRAVQSVSSSYLAWYQQKPGQAPRLLIYGASSRATGIPDRFSGSGSGTDFTLTISRLEPEDFAVYYCQQYGSSPTFGGGTKVEIK

SEQ ID NO: 9 - protein, 329 aa, Artificial sequence
  GPC3.1 heavy chain constant region
  MISC_FEATURE (97)..(97): R214 (EU/Kabat) allotype
  MISC_FEATURE (239)..(239): E356 (EU/Kabat) allotype
  MISC_FEATURE (241)..(241): M358 (EU/Kabat) allotype
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 107 aa, Homo sapiens [sequence omitted]

SEQ ID NO: 11 - protein, 446 aa, Artificial sequence
  GPC3.1 heavy chain
EVQLVQSGAEVKKPGESLKISCKGSGYSFTSYWIAWVRQMPGKGLEWMGIIFPGDSDTRYSPSFQGQVTISADRSIRTAYLQWSSLKASDTALYYCARTREGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, Homo Sapiens
  MISC_FEATURE: GPC3.1 kappa chain
EIVLTQSPGTLSLSPGERATLSCRAVQSVSSSYLAWYQQKPGQAPRLLIYGASSRATGIPDRFSGSGSGTDFTLTISRLEPEDFAVYYCQQYGSSPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1392 bp, Artificial Sequence
  GPC3.1 heavy chain with signal peptide
  sig_peptide (1)..(51)
  V_region (52)..(402)
  C_region (403)..(1392)
atgagggcttggatcttctttctgctctgcctggccgggagagcgctcgcagaggtgcagctggtgcagtctggagcagaggtgaaaaagcccggggagtctctgaagatctcctgtaagggttctggatacagctttaccagctactggatcgcctgggtgcgccagatgcccgggaaaggcctggagtggatggggatcatctttcctggtgactctgataccagatacagcccgtccttccaaggccaggtcaccatctcagccgacaggtccatcagaaccgcctacctgcagtggagcagcctgaaggcctcggacaccgccttgtattactgtgcgagaacccgggaggggtactttgactactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggttga

SEQ ID NO: 14 - protein, 463 aa, Artificial Sequence
  GPC3.1 heavy chain with signal peptide
  SIGNAL (1)..(17)
MRAWIFFLLCLAGRALAEVQLVQSGAEVKKPGESLKISCKGSGYSFTSYWIAWVRQMPGKGLEWMGIIFPGDSDTRYSPSFQGQVTISADRSIRTAYLQWSSLKASDTALYYCARTREGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - DNA, 696 bp, Artificial Sequence
  GPC3.1 kappa chain with signal peptide
  sig_peptide (1)..(51)
  V_region (52)..(372)
  C_region (373)..(696)
atgagggcttggatcttctttctgctctgcctggccgggcgcgccttggccgaaattgtgttgacgcagtctccaggcaccctgtctttgtctccaggggaaagagccaccctctcctgcagggccgttcagagtgttagcagcagctatttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 16 - protein, 231 aa, Artificial Sequence
  GPC3.1 kappa chain with signal peptide
  SIGNAL (1)..(17)
MRAWIFFLLCLAGRALAEIVLTQSPGTLSLSPGERATLSCRAVQSVSSSYLAWYQQKPGQAPRLLIYGASSRATGIPDRFSGSGSGTDFTLTISRLEPEDFAVYYCQQYGSSPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 17 aa, Homo sapiens
  MISC_FEATURE: Antibodies A and C heavy chain CDR2
IIFPGDSHTGYSPSFQG

SEQ ID NO: 18 - protein, 8 aa, Homo sapiens
  MISC_FEATURE: Antibody A heavy chain CDR3
TRQGYFDY

SEQ ID NO: 19 - protein, 8 aa, Homo sapiens
  MISC_FEATURE: Antibodies A and C kappa chain CDR3
QQYGSLPT

SEQ ID NO: 20 - protein, 5 aa, Homo sapiens
  MISC_FEATURE: Antibodies B and C heavy chain CDR1
RYWIA

SEQ ID NO: 21 - protein, 17 aa, Homo sapiens
  MISC_FEATURE: Antibody B heavy chain CDR2
IIFPGDSHTGYSPSFRG

SEQ ID NO: 22 - protein, 8 aa, Homo sapiens
  MISC_FEATURE: Antibody B kappa chain CDR3
QQYGRSPT

SEQ ID NO: 23 - protein, 117 aa, Homo sapiens
  MISC_FEATURE: Antibody A heavy chain variable region
EVQLVQSGAEVKKPGESLKISCKGSGYSFTSYWIAWVRQMPGKGLEWMGIIFPGDSHTGYSPSFQGQVTISADRSIRTAYLQWSSLKASDTAMYYCARTRQGYFDYWGQGTLVTVSS

SEQ ID NO: 24 - protein, 107 aa, Homo sapiens
  MISC_FEATURE: Antibody A kappa chain variable region
EIVLTQSPGTLSLSPGERATLSCRAVQSVSSSYLAWYQQKPGQAPRLLIYGASSRATGIPDRFSGSGSGTDFTLTISRLEPEDFAVYYCQQYGSLPTFGGGTKVEIK

SEQ ID NO: 25 - protein, 117 aa, Homo sapiens
  MISC_FEATURE: Antibody B heavy chain variable region
EVQLVQSGAEVKKPGESLKISCKGSGYSFTRYWIAWVRQMPGKGLEWMGIIFPGDSHTGYSPSFRGQVTISADKSISTAYLQWSSLKASDTAMYYCARTREGYFDYWGQGTLVTVSS

SEQ ID NO: 26 - protein, 107 aa, Homo sapiens
  MISC_FEATURE: Antibody B kappa chain variable region
EIVLTQSPGTLSLSPGERATLSCRAVQSVSSSYLAWYQQKPGQAPRLLIYGASSRATGIPDRFSGSGSGTDFTLTISRLEPEDFAVYYCQQYGRSPTFGGGTKVEIK

SEQ ID NO: 27 - protein, 117 aa, Homo sapiens
  MISC_FEATURE: Antibody C heavy chain variable region
EVQLVQSGAEVKKPGESLKISCKGSGYSFTRYWIAWVRQMPGKGLEWMGIIFPGDSHTGYSPSFQGQVTISADRSISTAYLQWSSLKASDTAMYYCARTREGYFDYWGQGTLVTVSS

SEQ ID NO: 28 - protein, 107 aa, Homo sapiens; identical to SEQ ID NO: 24
  MISC_FEATURE: Antibody C kappa chain variable region

(19) United States
(12) Patent Application Publication
(10) Pub. No.: US 2017/0326249 A1
(43) Pub. Date: Nov. 16, 2017

(54) ANTIBODY-DRUG CONJUGATE OF AN ANTI-GLYPICAN-3 ANTIBODY AND A TUBULYSIN ANALOG, PREPARATION AND USES

(71) Applicant: Bristol-Myers Squibb Company, Princeton, NJ (US)

(72) Inventors: Chin PAN, Los Altos, CA (US); Josephine M. CARDARELLI, San Carlos, CA (US); Haichun HUANG, Fremont, CA (US); Sanjeev GANGWAR, Foster City, CA (US); Qiang CONG, Palo Alto, CA (US); Karla A. HENNING, Milpitas, CA (US); Chetana RAO-NAIK, Walnut Creek, CA (US); Mohan SRINIVASAN, Cupertino, CA (US); Vangipuram S. RANGAN, Pleasant Hill, CA (US); Francis Y. LEE, Yardley, PA (US); Ginger C. RAKESTRAW, Somerville, MA (US)

(21) Appl. No.: 15/587,963

(22) Filed: May 5, 2017

Related U.S. Application Data
(60) Provisional application No. 62/333,944, filed on May 10, 2016.

(51) Int. Cl. A61K 47/68; C07K 16/30; A61K 9/00; C07K 5/02

(52) U.S. Cl. CPC A61K 47/6829; A61K 47/6849; C07K 5/0205; A61K 9/0019; C07K 16/303; C07K 2317/77; C07K 2317/92; C07K 2317/565; C07K 2317/56; C07K 2317/515; C07K 2317/51; C07K 2317/52

(57) ABSTRACT

An antibody-drug conjugate having a structure represented by formula (I)

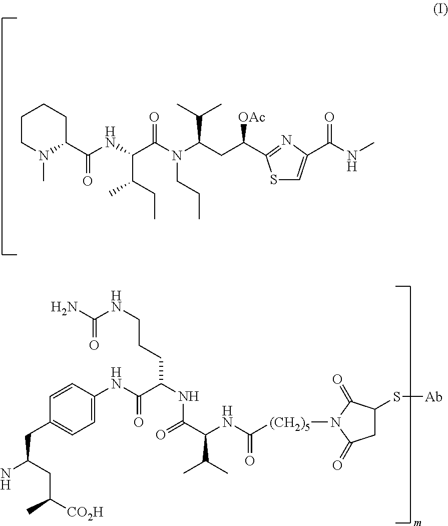

wherein m is 1, 2, 3, or 4 and Ab is an anti-glypican-3 antibody having heavy and light chain CDRs as disclosed herein.

### CROSS-REFERENCE TO RELATED APPLICATIONS

This application claims the benefit under 35 U.S.C. §119(e) of U.S. Provisional Application Ser. No. 62/333,944, filed May 10, 2016; the disclosure of which is incorporated herein by reference

### SEQUENCE LISTING

Incorporated herein by reference in its entirety is a Sequence Listing named “20170322_SEQT_12526USNP_YC.txt,” comprising SEQ ID NO:1 through SEQ ID NO:28, which include nucleic acid and/or amino acid sequences disclosed herein. The Sequence Listing has been submitted herewith in ASCII text format via EFS-Web, and thus constitutes both the paper and computer readable form thereof. The Sequence Listing was first created using Patent In 3.5 on Mar. 22, 2017, and is approximately 32 KB in size.

### BACKGROUND OF THE INVENTION

This disclosure relates to an antibody-drug conjugate of an anti-glypican-3 antibody and a tubulysin analog, and its preparation and uses.

Antibody-drug conjugates (ADCs, also referred to as immunoconjugates) are anti-cancer agents that are generating intense current interest. In an ADC, a therapeutic agent (also referred to as the drug, warhead, or payload) is covalently linked, or conjugated, to an antibody whose antigen is expressed by a cancer cell. The antibody, through its binding to the antigen, directs the ADC to the cancer—that is, the antibody acts as a targeting agent specifically delivering the ADC to the cancer cell. Once there, cleavage of the covalent link (referred to as the linker) or degradation of the antibody results in the release of the therapeutic agent at the cancer site. Conversely, while the ADC is circulating in the blood system, the therapeutic agent is held inactive because of its covalent linkage to the antibody. Thus, the therapeutic agent in an ADC can be much more potent (i.e., cytotoxic) than ordinary chemotherapy agents because of its localized release. Recently, two ADCs have received marketing approval: ADCETRIS™, in which an anti-CD30 antibody is conjugated to an auristatin, and KADCYLA™, in which the anti-Her2 antibody trastuzumab is conjugated to a maytansinoid. For a review on the mode of action of ADCs, see Schrama et al. 2006. (The full bibliographic citation for this and other documents cited herein by first author or inventor and year are listed at the end of this specification.)

Glypican-3 is an oncofetal antigen that belongs to the glypican family of glycosyl-phosphatidylinositol-anchored heparin sulfate proteoglycans. Glypicans are characterized by a covalent linkage to complex polysaccharide chains called heparinsulphate glycosaminoglycans. Glypicans are involved in cell signaling at the cellular-extracellular matrix interface (Sasisekharan et al. 2002). To date, six distinct members of the human glypican family have been identified. Cell membrane-bound glypican-3 is composed of two subunits, linked by one or more disulfide bonds.

Glypican-3 is expressed in fetal liver and placenta during development and is down-regulated or silenced in normal adult tissues. Mutations and depletions in the glypican-3 gene are responsible for the Simpson-Golabi-Behmel or Simpson dysmorphia syndrome in humans. Glypican-3 is expressed in various cancers and, in particular, hepatocellular carcinoma (HCC, the most common form of liver cancer), melanoma, Wilm's tumor, and hepatoblastoma (Jakubovic and Jothy 2007; Nakatsura and Nishimura 2005).

HCC is the third leading cause of cancer-related deaths worldwide. Each year, HCC accounts for about 1 million deaths (Nakatsura and Nishimura 2005). Hepatitis B virus, hepatitis C virus, and chronic heavy alcohol use leading to cirrhosis of the liver remain the most common causes of HCC. Its incidence has increased dramatically in the United States because of the spread of hepatitis C virus infection and is expected to increase for the next 2 decades. HCC is treated primarily by liver transplantation or tumor resection. Patient prognosis is dependent on both the underlying liver function and the stage at which the tumor is diagnosed (Parikh and Hyman 2007). Thus, effective HCC treatment strategies are needed.

There are various disclosures of the uses of anti-glypican-3 antibodies in cancer therapy, either as a therapeutic antibody or in a conjugate. Smith et al. 2007 disclose conjugates of an anti-glypican-3 antibody and an auristatin. Zhang et al. 2014 disclose ADCs of an anti-glypican-3 antibody and a DNA minor groove binder-alkylator of the cyclopropabenzindole (CBI) type. Terrett et al. 2014 disclose anti-glypican-3 antibodies and their use for treating glypican-3 related conditions, including HCC and, generically, their use in immunoconjugates. Other disclosures relating to immunoconjugates of anti-glypican-3 antibodies include Ho et al., 2015 and 2015.

### BRIEF SUMMARY OF THE INVENTION

This disclosure provides an antibody-drug conjugate (ADC) comprising an anti-glypican-3 antibody as the targeting agent and a tubulysin analog, which ADC has an unexpectedly desirable combination of potency, therapeutic index, and pharmacokinetic properties and which can be used to treat a variety of cancers, including HCC, lung cancer, and ovarian cancer. The antibody-drug conjugate has a structure represented by formula I

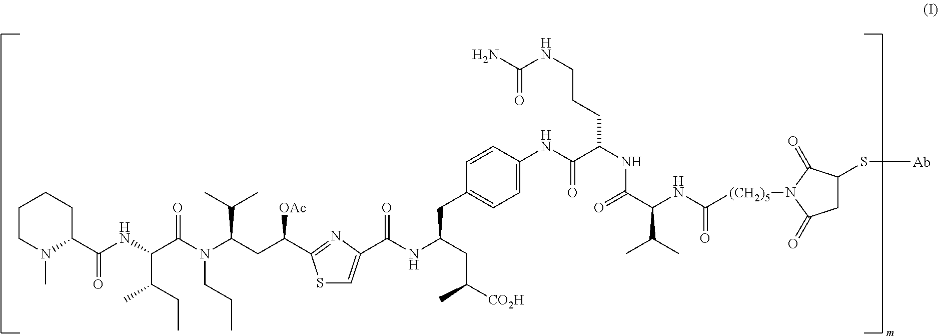

wherein
m is 1, 2, 3, or 4 and
Ab is an anti-glypican-3 antibody having
   (a) a heavy chain variable region CDR1 comprising SEQ ID NO:1;
   (b) a heavy chain variable region CDR2 comprising SEQ ID NO:2;
   (c) a heavy chain variable region CDR3 comprising SEQ ID NO:3;
   (d) a kappa light chain variable region CDR1 comprising SEQ ID NO:4;
   (e) a kappa light chain variable region CDR2 comprising SEQ ID NO:5; and
   (f) a kappa light chain variable region CDR3 comprising SEQ ID NO:6.

As reflected by the subscript m, each antibody Ab can conjugate with more than one drug moiety, depending on the number of sites antibody Ab has available for conjugation and the experimental conditions employed. Those skilled in the art will appreciate that, while each individual antibody Ab is conjugated to an integer number of drug moieties, a conjugate preparation of the conjugate may analyze for a non-integer ratio of drug moieties to antibody Ab, reflecting a statistical average. This ratio is referred to as the substitution ratio (SR) or, synonymously, the drug-antibody ratio (DAR). Preferably, each antibody Ab is conjugated to 3 or 4 drug moieties (i.e., m is 3 or 4). The average m for a conjugate preparation preferably is between 3 and 3.5 (i.e., the DAR is 3 to 3.5).

In a preferred embodiment, the antibody Ab has a heavy chain variable region amino acid sequence according to SEQ ID NO:7 and a kappa light chain variable region amino acid sequence according to SEQ ID NO:8.

In another preferred embodiment, antibody Ab has a heavy chain constant region comprising comprising SEQ ID NO:9 and a kappa light chain constant region comprising SEQ ID NO:10. The heavy chain constant region may further have a lysine at its C-terminus.

In yet another preferred embodiment, antibody Ab has a heavy chain comprising SEQ ID NO:11 and a kappa light chain comprising SEQ ID NO:12. Such antibody is referred to herein as GPC3.1. Correspondingly, the ADC of formula I where the antibody is GPC3.1 is referred to herein as ADC3.1.

The heavy chain of antibody GPC3.1 optionally may further have a lysine at its C-terminus.

This disclosure also provides a method of treating a cancer in a human subject suffering from such cancer, comprising administering to the human subject a therapeutically effective amount of an antibody-drug conjugate of this disclosure, where the cancer is hepatocellular carcinoma, ovarian, or lung cancer, especially liver or lung cancer. The antibody-drug conjugate preferably is administered intravenously, at a dose of between 0.1 and 20 mg/kg, preferably between 0.5 and 15 mg/kg, and more preferably between 1.0 and 5 mg/kg.

This disclosure also provides a pharmaceutical formulation comprising an antibody-drug conjugate of this disclosure and a pharmaceutically acceptable excipient.

This disclosure also provides an isolated nucleic acid molecule encoding an antibody heavy chain comprising SEQ ID NO:11, which nucleic acid molecule preferably comprises SEQ ID NO:13.

This disclosure also provides an expression vector comprising the nucleic acid molecule of SEQ ID NO:13, and a host cell comprising such expression vector.

This disclosure also provides an isolated nucleic acid molecule encoding an antibody kappa chain comprising SEQ ID NO:12, which nucleic acid molecule comprises SEQ ID NO:15.

This disclosure also provides an expression vector comprising the nucleic acid molecule of SEQ ID NO:15, and a host cell comprising such expression vector.

### BRIEF DESCRIPTION OF THE DRAWING(S)

FIGS. 1A-1C show in combination the alignment between a nucleotide sequence encoding a signal peptide and the heavy chain of an anti-glypican-3 antibody of this disclosure (SEQ ID NO:13) and the amino acid sequence so encoded (SEQ ID NO:14).

FIGS. 2A-2B show in combination the alignment between a nucleotide sequence encoding a signal peptide and the kappa light chain of an anti-glypican-3 antibody of this disclosure (SEQ ID NO:15) and the amino acid sequence so encoded (SEQ ID NO:16).

FIGS. 3A-3C show, in combination, that antibody GPC3.1 binds to Hep3B hepatocellular carcinoma and H446 small-cell lung cancer cells, and that, when conjugated to a drug, it effectively delivers the drug to inhibit proliferation of such cells.

FIGS. 4A-4B show the dose dependent efficacy of ADC3.1 in a Hep3B xenograft model, as measured by tumor volume shrinkage and per cent body weight change, respectively.

FIGS. 5A-5B and 6A-6B compare the efficacy of ADC3.1 in single dosing and split dosing administration regimens, against HuH7D12 hepatocellular carcinoma tumors.

FIGS. 7A-7B and 8A-8B compare the efficacy of ADC3.1 in single dosing and split dosing administration regimens, against H446 small-cell lung cancer tumors.

FIGS. 9 and 10 are xenograft studies showing the efficacy of ADC3.1 against ovarian and squamous lung patient derived tumor cells, respectively.

FIGS. 11A-11C compare the properties of antibody GPC3.1 and three anti-glypican-3 antibodies that are variants of antibody GPC3.1, and of their respective ADCs.

FIG. 12A compares the pharmacokinetic and in vitro properties of GPC3.1 against those of other anti-glypican-3 antibodies prepared by independent immunizations of transgenic mice. FIG. 12B compares the in vitro potencies of ADC3.1 and an ADC prepared from one of those other antibodies.

FIGS. 13A-13B compare the in vivo efficacy of ADC3.1 against those of ADCs made with other anti-glypican-3 antibodies.

### DETAILED DESCRIPTION OF THE INVENTION

### Definitions

“Antibody” means whole antibodies and any antigen binding fragment (i.e., “antigen-binding portion”) or single chain variants thereof. A whole antibody is a protein comprising at least two heavy (H) chains and two light (L) chains inter-connected by disulfide bonds. Each heavy chain comprises a heavy chain variable region (VH) and a heavy chain constant region comprising three domains, C_{H1}, C_{H2} and C_{H3}. Each light chain comprises a light chain variable region (V_{L} or Vₖ) and a light chain constant region comprising one single domain, C_{L}. The V_{H} and V_{L} regions can be further subdivided into regions of hypervariability, termed complementarity determining regions (CDRs), interspersed with more conserved framework regions (FRs). Each V_{H} and V_{L} comprises three CDRs and four FRs, arranged from amino- to carboxy-terminus in the following order: FR1, CDR1, FR2, CDR2, FR3, CDR3, and FR4. The variable regions contain a binding domain that interacts with an antigen. The constant regions may mediate the binding of the antibody to host tissues or factors, including various cells of the immune system (e.g., effector cells) and the first component (Clq) of the classical complement system. An antibody is said to “specifically bind” to an antigen X if the antibody binds to antigen X with a K_{D} of 5×10⁻⁸ M or less, more preferably 1×10⁻⁸ M or less, more preferably 6×10⁻⁹ M or less, more preferably 3×10⁻⁹ M or less, even more preferably 2×10⁻⁹ M or less. The antibody can be chimeric, humanized, or, preferably, human. The heavy chain constant region can be engineered to affect glycosylation type or extent, to extend antibody half-life, to enhance or reduce interactions with effector cells or the complement system, or to modulate some other property. The engineering can be accomplished by replacement, addition, or deletion of one or more amino acids or by replacement of a domain with a domain from another immunoglobulin type, or a combination of the foregoing.

“Antigen binding fragment” and “antigen binding portion” of an antibody (or simply “antibody portion” or “antibody fragment”) mean one or more fragments of an antibody that retain the ability to specifically bind to an antigen. It has been shown that the antigen-binding function of an antibody can be performed by fragments of a full-length antibody, such as (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fab′ fragment, which is essentially an Fab with part of the hinge region (see, for example, Abbas et al., Cellular and Molecular Immunology, 6th Ed., Saunders Elsevier 2007); (iv) a Fd fragment consisting of the V_{H} and C_{H1} domains; (v) a Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Preferred antigen binding fragments are Fab, F(ab′)₂, Fab′, Fv, and Fd fragments. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv, or scFv); see, e.g., Bird et al. (1988) Science 242:423-426; and Huston et al. (1988) Proc. Natl. Acad. Sci. USA 85:5879-5883). Such single chain antibodies are also encompassed within the term “antigen-binding portion” of an antibody.

An “isolated antibody” means an antibody that is substantially free of other antibodies having different antigenic specificities (e.g., an isolated antibody that specifically binds antigen X is substantially free of antibodies that specifically bind antigens other than antigen X). An isolated antibody that specifically binds antigen X may, however, have cross-reactivity to other antigens, such as antigen X molecules from other species. In certain embodiments, an isolated antibody specifically binds to human antigen X and does not cross-react with other (non-human) antigen X antigens. Moreover, an isolated antibody may be substantially free of other cellular material and/or chemicals.

“Monoclonal antibody” or “monoclonal antibody composition” means a preparation of antibody molecules of single molecular composition, which displays a single binding specificity and affinity for a particular epitope.

“Human antibody” means an antibody having variable regions in which both the framework and CDR regions (and the constant region, if present) are derived from human germ-line immunoglobulin sequences. Human antibodies may include later modifications, including natural or synthetic modifications. Human antibodies may include amino acid residues not encoded by human germline immunoglobulin sequences (e.g., mutations introduced by random or site-specific mutagenesis in vitro or by somatic mutation in vivo). However, “human anti-body” does not include antibodies in which CDR sequences derived from the germline of another mammalian species, such as a mouse, have been grafted onto human framework sequences.

“Human monoclonal antibody” means an antibody displaying a single binding specificity, which has variable regions in which both the framework and CDR regions are derived from human germline immunoglobulin sequences. In one embodiment, human monoclonal antibodies are produced by a hybridoma that includes a B cell obtained from a transgenic nonhuman animal, e.g., a transgenic mouse, having a genome comprising a human heavy chain transgene and a light chain transgene fused to an immortalized cell.

### Antibody GPC3.1

At a first glance, the role to be performed by an antibody in an ADC appears to be a simple one: lead the conjugated drug to the target cell, and once there, release its drug payload, either inside the target cell or in the environs thereof. However, the selection of a suitable antibody for a successful ADC entails many variables beyond merely binding to the antigen of interest. Multiple factors may affect the overall efficacy of an ADC, including stability in circulation prior to reaching the target cell, binding affinity the antigen, safety vis-a-vis non-target cells that also express the antigen, and pharmacokinetics. The interplay among these factors is difficult to predict. As the data presented hereinbelow demonstrates, not all antibodies binding to glypican-3 produce an ADC as efficacious as antibody GPC3.1.

CDR1, CDR2, and CDR3 of the heavy chain of antibody GPC3.1 comprise the amino acids of SEQ ID NO:1, SEQ ID NO:2, and SEQ ID NO:3, respectively. CDR1, CDR2, and CDR3 of the light (kappa) chain of antibody GPC3.1 comprise the amino acids of SEQ ID NO:4, SEQ ID NO:5, and SEQ ID NO:6, respectively. The amino acid sequences of the heavy and kappa chain variable regions are given by SEQ ID NO:7 and SEQ ID NO:8, respectively.

The heavy chain constant region of antibody GPC3.1 is of the IgG1 isotype, comprising the R214, E356, and M358 allotypes (numbering per EU index as set forth in Kabat et al., “Sequences of proteins of immunological interest, 5th ed., Pub. No. 91-3242, U.S. Dept. Health & Human Services, NIH, Bethesda, Md., 1991; hereinafter “Kabat”). Its amino acid sequence is set forth in SEQ ID NO:9. This allotype combination has a high prevalence in the Caucasian population.

The kappa light chain constant region of antibody GPC3.1 has an amino acid sequence as set forth in SEQ ID NO:10.

The complete heavy and kappa light chain amino acid sequences of antibody GPC3.1 are set forth in SEQ ID NO:11 and NO:12, respectively.

Terrett et al. 2014 disclose an anti-glypican-3 antibody 4A6 that has the same heavy and light chain variable regions as antibody GPC3.1, of the IgG1 or IgG4 isotype. It further generically discloses that antibody 4A6 can be used in ADCs, but does not provide any working examples.

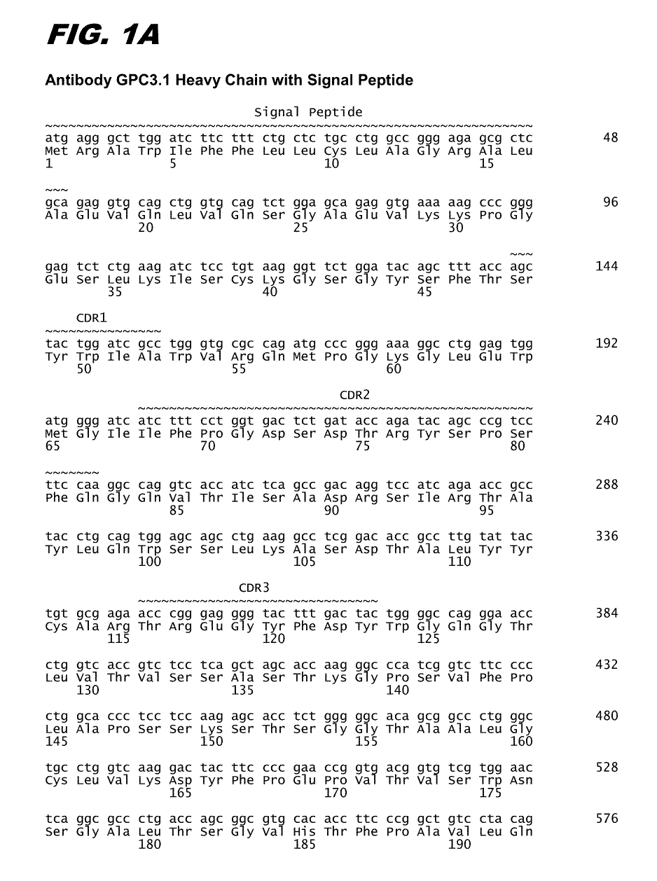

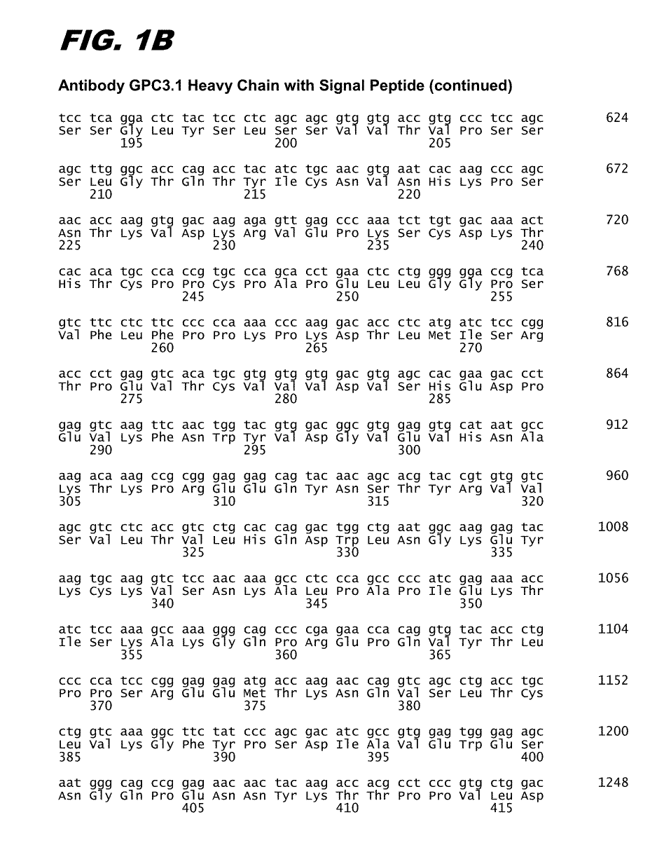

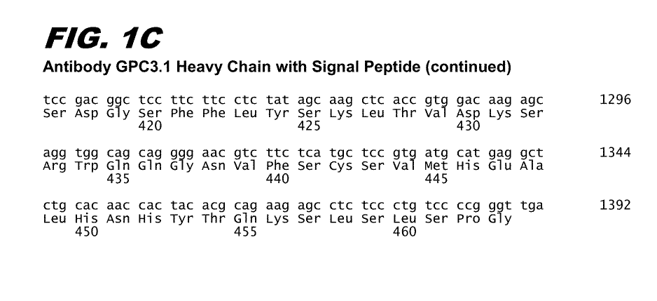

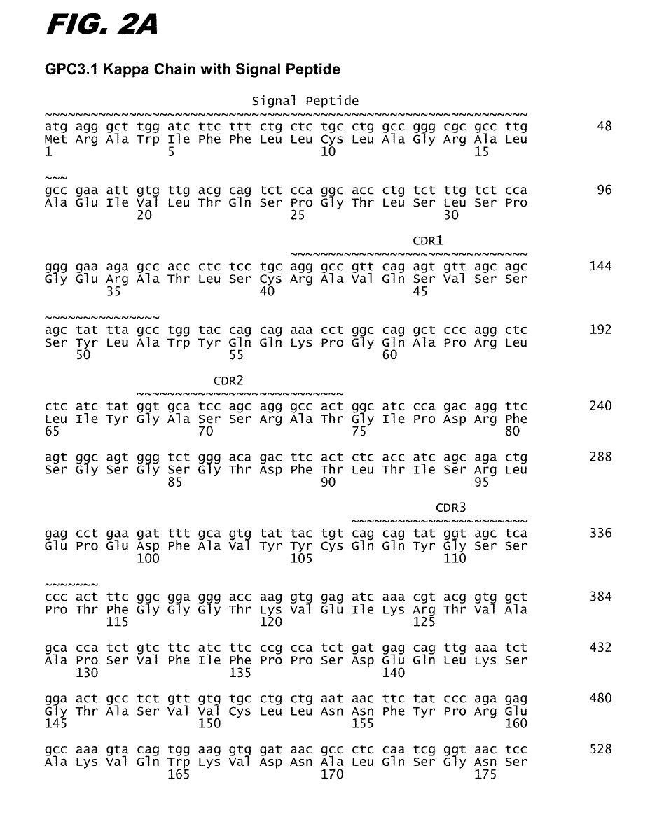

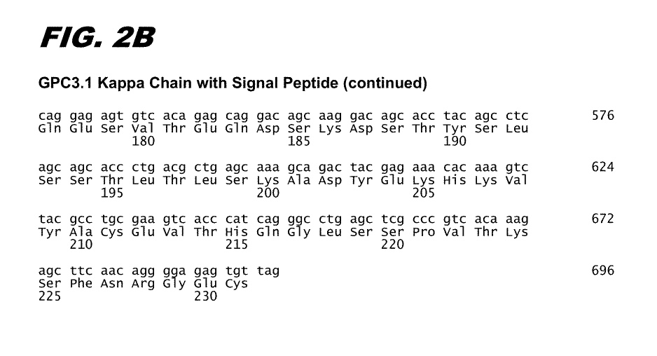

Antibody GPC3.1 can be produced by recombinant expression of its heavy and kappa chains in a suitable host cell. SEQ ID NO:13 shows a DNA sequence, inclusive of a signal peptide, that can be used for recombinant production of the heavy chain, while SEQ ID NO:14 shows the amino acid sequence encoded thereby. The alignment between the DNA and amino acid sequences is shown in FIGS. 1A-1C. SEQ ID NO:15 shows a DNA sequence, inclusive of a signal peptide, that can be used for recombinant production of the kappa chain, while SEQ ID NO:16 shows the amino acid sequence encoded thereby. The alignment between the DNA and amino acid sequences is shown in FIGS. 2A-2B.

Those skilled in the art will know that, when an antibody is produced recombinantly with a heavy chain C-terminal lysine group, such lysine is often removed by endogenous carboxypeptidases during cell culture production (Luo et al. 2012). Therefore antibody GPC3.1 can also be produced employing a DNA sequence corresponding to SEQ ID NO:13 but with an added codon for lysine at the C-terminal position and then allowing post-translational enzymatic removal of the lysine.

This disclosure also provides nucleic acids encoding antibody GPC3.1, in particular a nucleic acid (SEQ ID NO:13) encoding its heavy chain (SEQ ID NO:11), and conservative modifications of such nucleic acids. A “conservative modification” means, in respect of a nucleic acid sequence, a modification that replaces a nucleic acid therein with another but the modification results in the modified nucleic acid sequence encoding the same or a conservatively modified amino acid sequence compared to the one encoded by the original nucleic acid sequence or, where the original nucleic acid does not encode an amino acid sequence, the resultant modified nucleic acid sequence is essentially the same as the original nucleic acid sequence. Because of the degeneracy of the genetic code, a large number of functionally identical nucleic acid sequences may encode any given protein. A nucleic acid sequence may have plural conservative modifications.

Where a polypeptide or nucleic acid molecule is associated with a particular SEQ ID NO:, preferably such polypeptide or nucleic acid molecule consists of the amino acid or nucleic acid sequence of the associated SEQ ID NO:.

### Therapeutic Agent and Linker

The therapeutic agent in the conjugate of this disclosure is a synthetic tubulysin analog and has a structure represented by formula (II) (Cheng et al. 2013):

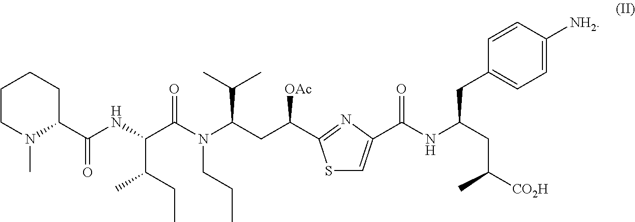 [SMILES: CCCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(N)cc2)C[C@H](C)C(=O)O)cs1)C(C)C]

The tubulysins are potent naturally occurring cytotoxins, which act as anti-mitotic agents that interfere with mitosis by preventing the assembly of the tubulins into microtubules. The affected cells to accumulate in the G₂/M phase and undergo apoptosis.

To conjugate a therapeutic agent to an antibody, a linker moiety is needed. In the instance of the present invention, the linker moiety has a structure represented by formula (III):

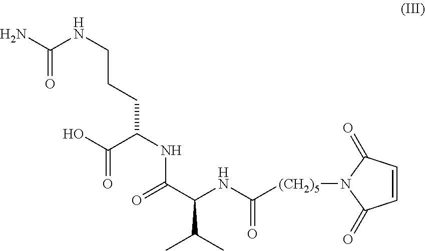

It comprises a valine-citrulline dipeptide (Val-Cit, recited in the conventional N-to-C direction), which is designed to be cleaved by the intracellular enzyme cathepsin B after the ADC has reached a target cancer cell and has been internalized by it, thus releasing the therapeutic agent to exert its cytotoxic effect. See Dubowchik et al. 1998a, 1998b, and 2002.

In the preparation of the conjugate of this disclosure, drug (II) and linker (III) are coupled to produce a therapeutic agent-linker compound having a structure represented by formula (IV), by forming an amide bond between the —CO₂H group of the citrulline in linker (II) and the aromatic —NH₂ of compound (II).

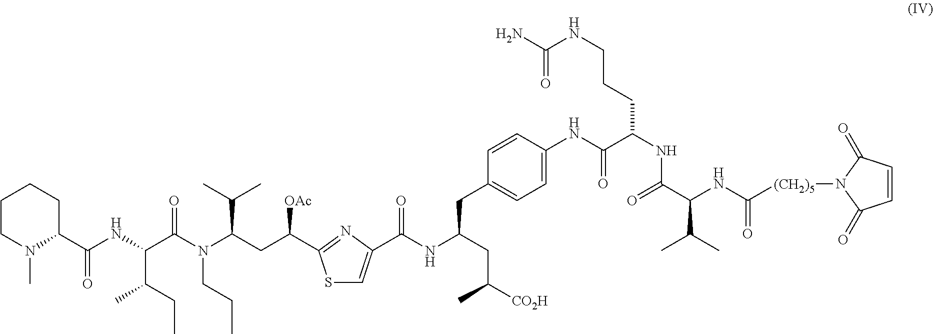

Compound (IV) is then conjugated to the antibody to prepare an ADC of formula (I). An ε-amino group in the side chain of a lysine residue of antibody GPC3.1 is reacted with 2-iminothiolane to introduce a free thiol (—SH) group. The thiol group can react with the maleimide group in compound (IV) to effect conjugation:

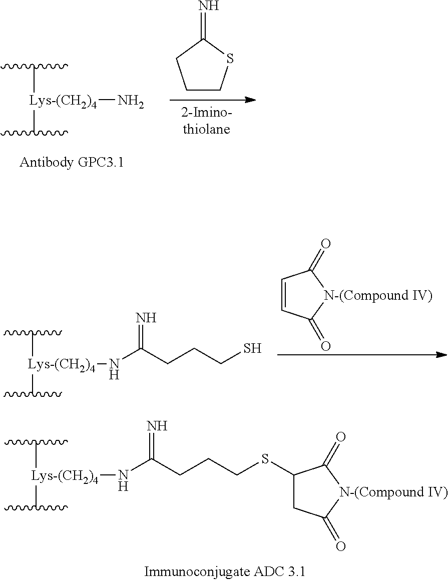

Typically, a thiolation level of two to four thiols per antibody is achieved. For a representative procedure, see Cong et al. 2015, the disclosure of which is incorporated herein by reference.

In addition to the naturally occurring tubulysins, synthetic tubulysin analogs with potent cytotoxic activity are known, for example as disclosed in Cheng et al. 2013 and Cong 2015. These references further disclose that such tubulysin analogs can be used in ADCs.

In particular, Cheng et al. 2013 discloses the preparation of a tubulysin analog-linker compound referred to there as formula (VI-t) (i.e., identical to formula (IV) above excepting the racemic methyl group alpha to the carboxylic acid) and, at Table 4 therein, its conjugates with an anti-CD70 antibody or anti-mesothelin antibody.

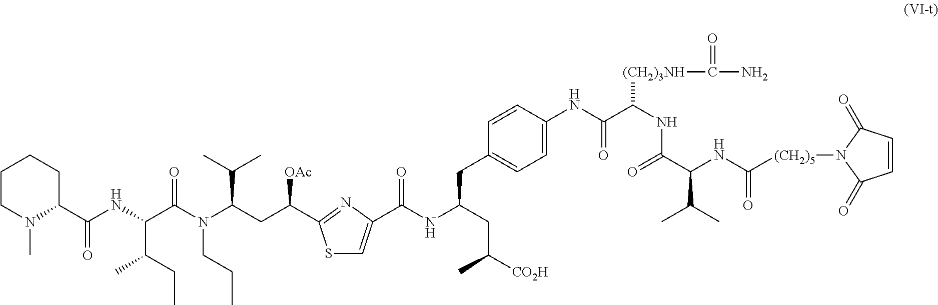

### EXAMPLES

The practice of this invention can be further understood by reference to the following examples, which are provided by way of illustration and not of limitation.

### Example 1

The antibody GPC3.1 V_{H} and V_{K} sequences were cloned into expression vectors containing the osteonectin signal sequence and the human IgG1 and kappa constant regions. The resulting heavy and light chain expression vectors were co-transfected into CHO cells and stable clones were selected and screened for IgG expression. One clone was chosen and expanded for antibody production.

### Example 2

This general procedure can be used to make ADC3.1 and other antibody-drug conjugates disclosed herein. Initially the antibody is buffer exchanged into 0.1 M phosphate buffer (pH 8.0) containing 50 mM NaCl and 2 mM diethylene triamine pentaacetic acid (DTPA) and concentrated to 5-10 mg/mL. Thiolation is achieved through addition of 2-iminothiolane to the antibody. The amount of 2-iminothiolane to be added can be determined by a preliminary experiment and varies from antibody to antibody. In the preliminary experiment, a titration of increasing amounts of 2-iminothiolane is added to the antibody, and following incubation with the antibody for 1 h at RT (room temperature, circa 25° C.), the antibody is desalted into 50 mM HEPES, 5 mM Glycine, 2 mM DTPA, pH 5.5 using a SEPHADEX™ G-25 column and the number of thiol groups introduced determined rapidly by reaction with dithiodipyridine (DTDP). Reaction of thiol groups with DTDP results in liberation of thiopyridine, which can be monitored spectroscopically at 324 nm. Samples at a protein concentration of 0.5-1.0 mg/mL are typically used. The absorbance at 280 nm can be used to accurately determine the concentration of protein in the samples, and then an aliquot of each sample (0.9 mL) is incubated with 0.1 mL DTDP (5 mM stock solution in ethanol) for 10 min at RT. Blank samples of buffer alone plus DTDP are also incubated alongside. After 10 min, absorbance at 324 nm is measured and the number of thiol groups is quantitated using an extinction coefficient for thiopyridine of 19,800 M⁻¹.

Typically a thiolation level of about two to three thiol groups per antibody is desirable. For example, with some antibodies this can be achieved by adding a 15-fold molar excess of 2-iminothiolane followed by incubation at RT for 1 h. The antibody is then incubated with 2-iminothiolane at the desired molar ratio and then desalted into conjugation buffer (50 mM HEPES, 5 mM glycine, 2 mM DTPA, pH 5.5)). The thiolated material is maintained on ice while the number of thiols introduced is quantitated as described above.

After verification of the number of thiols introduced, the dimer-linker of formula (IV) is added at a 2.5-fold molar excess per thiol. The conjugation reaction is allowed to proceed in conjugation buffer containing a final concentration of 25% propylene glycol and 5% trehalose. Commonly, the drug-linker stock solution is dissolved in 100% DMSO. The stock solution is added directly to the thiolated antibody.

The conjugation reaction mixture is incubated at RT for 2 h with gentle stirring. A 10-fold molar excess of N-ethyl maleimide (100 mM stock in DMSO) is then added to the conjugation mixture and stirred for an additional hour to block any unreacted thiols.

The sample is then filtered via a 0.2μ filter The material is buffer exchanged via TFF VivaFlow 50 Sartorius 30 MWCO PES membrane into 10 mg/mL glycine, 20 mg/mL sorbitol, 15% acetonitrile (MeCN) pH 5.0 (5× TFF buffer exchange volume), to remove any unreacted drug. The final formulation is carried out by TFF into 20 mg/mL sorbitol, 10 mg/mL glycine, pH 5.0.

### Example 3

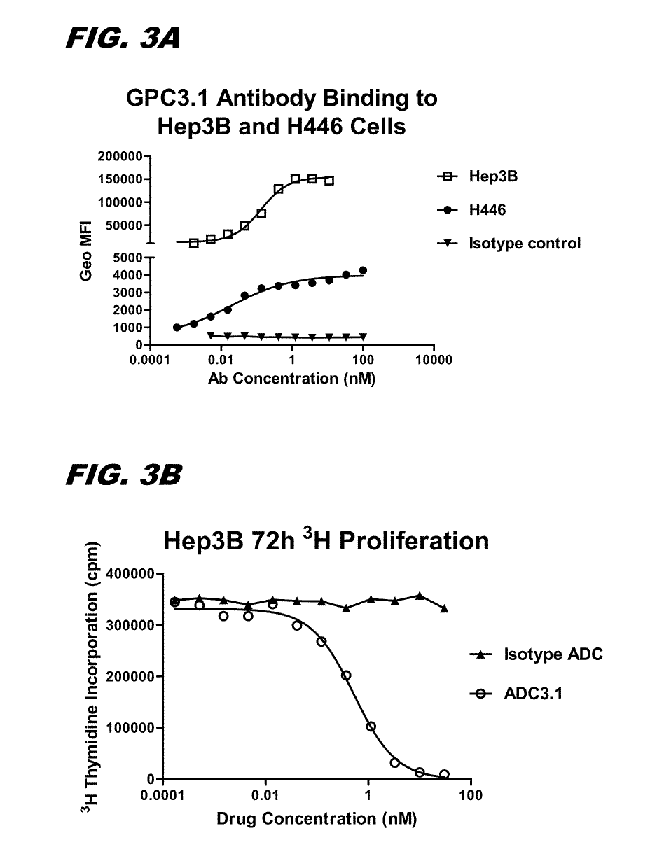

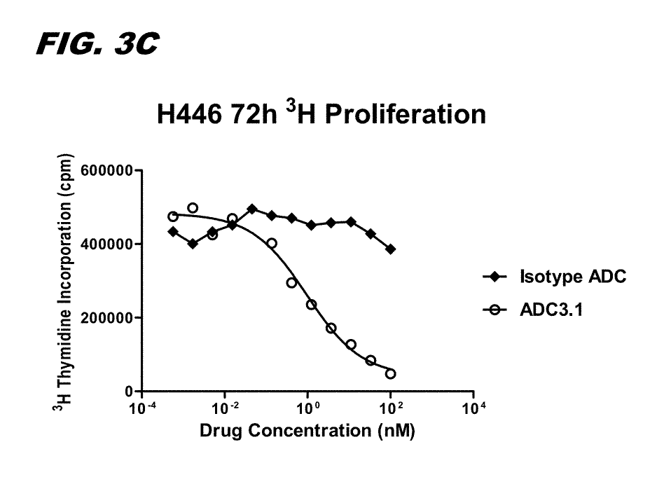

FIG. 3A compares the binding of ADC3.1 to Hep3B hepatocellular carcinoma (liver cancer) and H446 small-cell lung cancer (SCLC) cells. The higher binding to Hep3B cells indicates that they express a higher level of glypican-3 than H446 cells. The isotype control was an anti-CD70 antibody.

A ³H thymidine assay, where the inhibition of incorporation of ³H thymidine indicates inhibition of proliferation of the tested cell line, was used to assess the dose-dependent inhibitory effect of ADC3.1 on the proliferation of Hep3B and H446 cells. The human tumor cell lines were obtained from the American Type Culture Collection (ATCC), P.O. Box 1549, Manassas, Va. 20108, USA, and cultured according to instructions from ATCC. Cells were seeded at 1.0×10⁴ cells/well in 96-well plates. 1:3 serial dilutions of ADC3.1 were added to the wells. Plates were allowed to incubate for 72 h. The plates were pulsed with 1.0 μCi of ³H-thymidine per well for the last 24 hours of the total incubation period, harvested, and read on a Top Count Scintillation Counter (Packard Instruments, Meriden, Conn.). The EC₅₀ values—the ADC concentration at which cell proliferation was reduced by 50%—were determined using PRISM™ software, version 4.0 (GraphPad Software, La Jolla, Calif., USA).

FIGS. 3B and 3C show that ADC3.1 inhibits proliferation of the tested cell lines in a dose-dependent manner, while the isotype ADC (control) had essentially no inhibitory effect. In FIG. 3B the control was a conjugate of an anti-mesothelin antibody and the drug-linker compound of formula (IV). In FIG. 3C the control was a conjugate of an anti-CD70 antibody and the drug-linker compound of formula (IV).

The EC₅₀ so values, along with those against HepG2 and HuH7D12 hepatocellular carcinoma cell lines, are shown in Table 1, plus data for the drug alone (formula (II)). These results show that, generally, ADC3.1 is effective in delivering the drug to the target cell, at a level comparable to that of unconjugated drug. That is, release of the drug from the conjugate is efficient.

TABLE 1
Efficacy of ADC Compared to Unconjugated Drug

| | EC₅₀ (nM) | | | |
|---|---|---|---|---|
| Agent | Hep3B | HepG2 | HuH7D12 | H446 |
| ADC3.1 | 0.2 | 0.3 | 0.2 | 1.0 |
| Unconjugated Drug (II) | 0.2 | 0.3 | 0.2 | 0.2 |

### Example 4

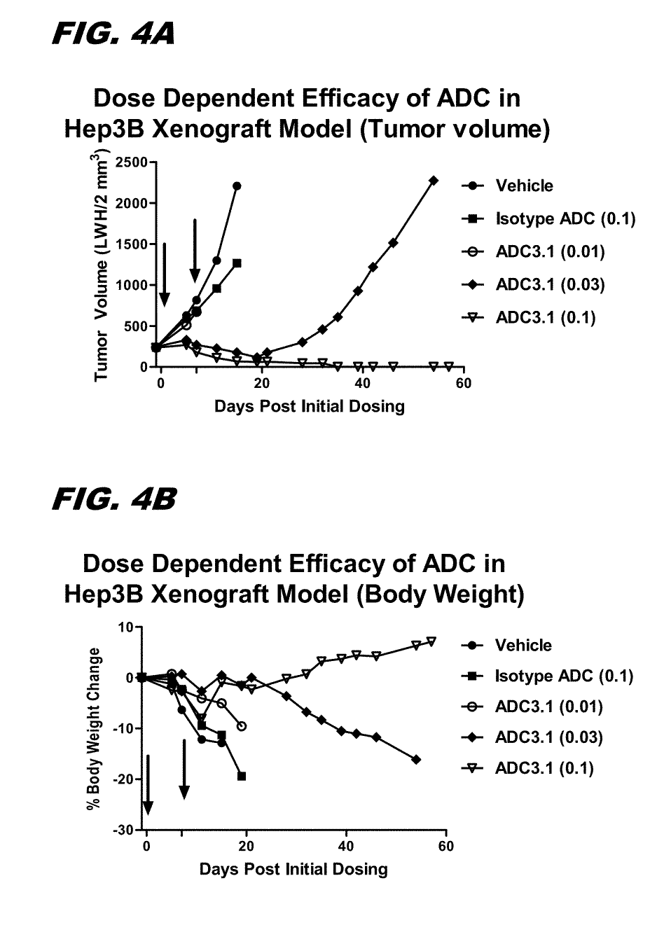

FIGS. 4A and 4B present the data for a xenograft study on the dose dependent efficacy of ADC3.1 against Hep3B tumors. ADC3.1 was administered twice, seven days apart (Q7D×2), at doses of 0.01, 0.03, and 0.1 μmol/kg. (A dose of 0.1 μmol/kg corresponds approximately to 5 mg/kg. Thus, the dosages convert to 0.5, 1.5, and 5 mg/kg, respectively.) The isotype ADC was the same as in FIG. 3C. In this and other xenograft studies described herein, CB17. SCID mice were used.

The data show that, at 0.1 μmol/kg, ADC3.1 was highly effective in causing tumor regression (FIG. 4A), was well tolerated, and relieved tumor growth related cachexia. The vehicle (formulation buffer) and ADC isotype controls were ineffective, with the selectivity between ADC3.1 and the isotype control being greater than 3:1. Lower doses of ADC3.1 (0.01 and 0.03 μmol/kg) were significantly less effective. This steep dose dependent response is believed to be partially due to the nonlinear pharmacokinetics of ADC3.1.

### Example 5

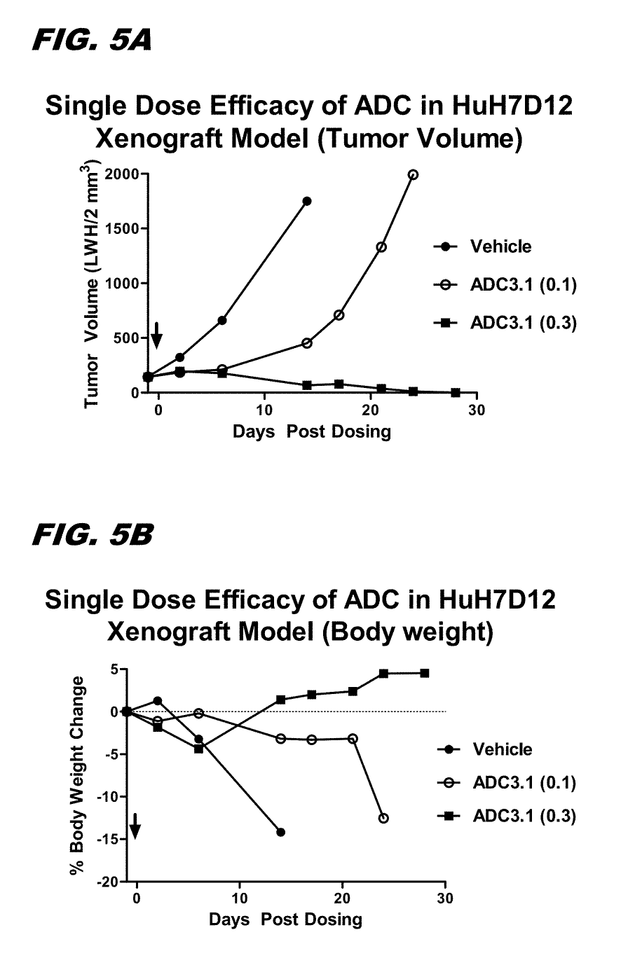

The efficacy of ADC3.1 in a xenograft study of the single does efficacy of ADC3.1 against HuH7D12 cells is shown in FIG. 5A (tumor volume regression) and FIG. 5B (per cent body weight change). The dosages were 0.1 and 0.3 μmol/kg (5 and 15 mg/kg, respectively).

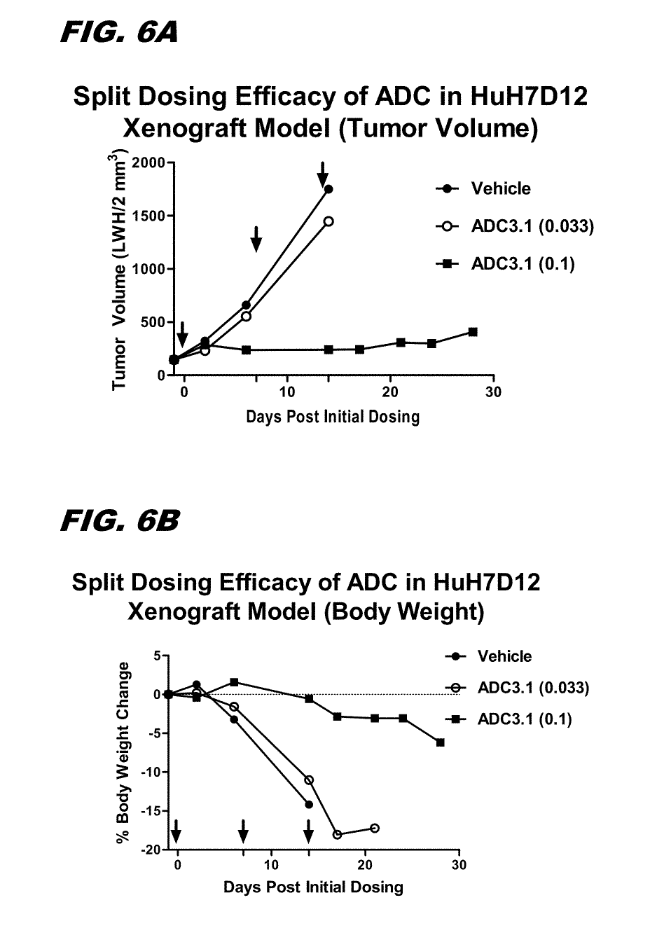

The corresponding split dosing study is shown in FIGS. 6A and 6B, with three doses administered in one-week intervals (Q7D×3). The doses were 0.033 and 0.1 μmol/kg.

The results indicate that the single dosing regimen was more efficacious, with tumor regression noted with a single dose of 0.3 μmol/kg (seven of eight mice becoming tumor-free).

### Example 6

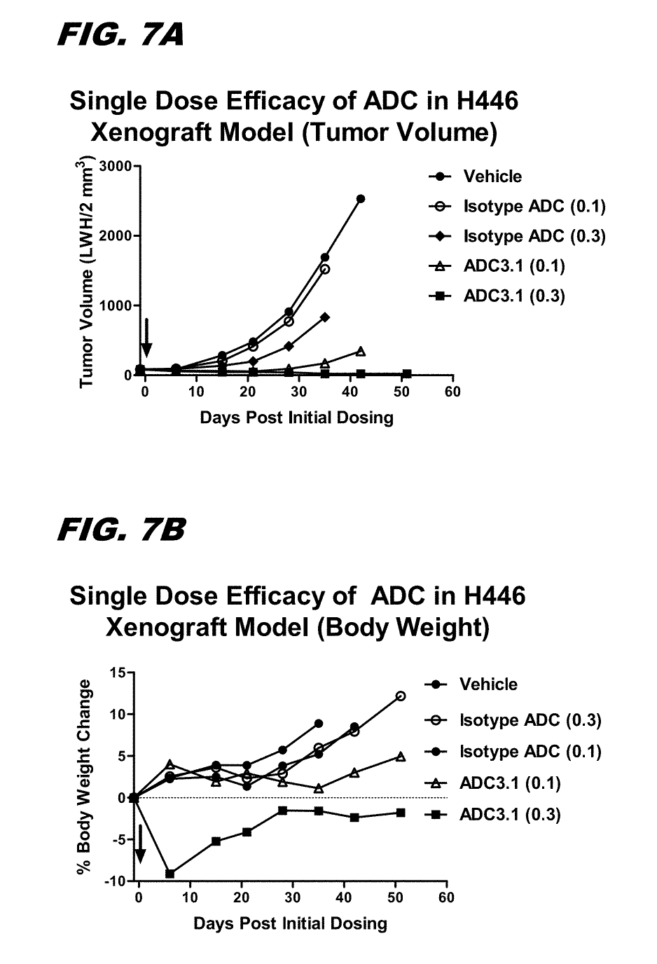

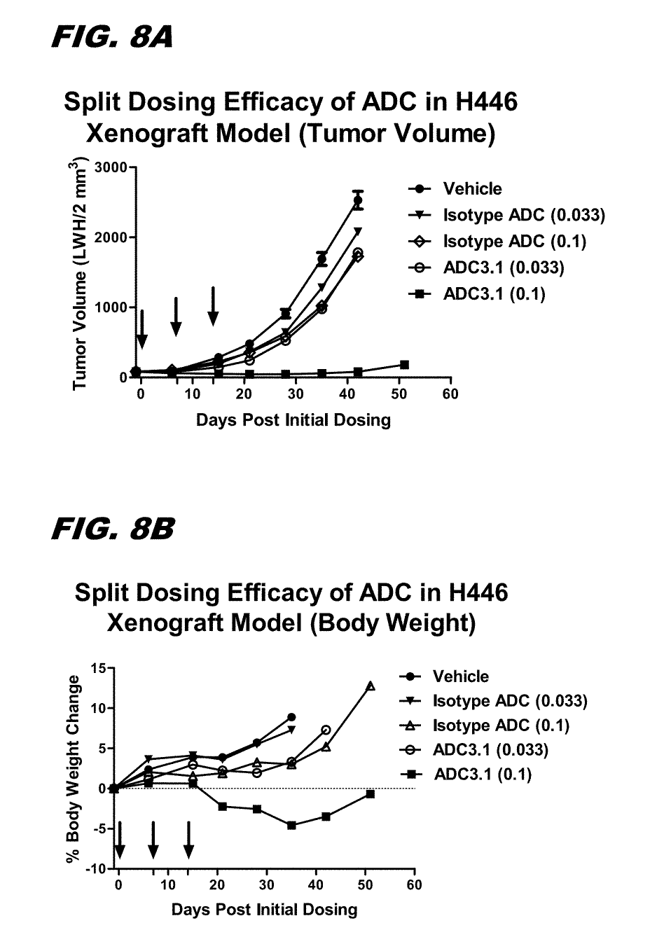

A similar xenograft study was performed, comparing single dose (FIGS. 7A-7B) and split dose (Q7D×3, FIGS. 8A-8B) administration schedules on the efficacies of ADC3.1 against H446 cells. The doses amounts noted parenthetically in the figures are in μmol/kg, corresponding to 5 and 15 mg/kg, respectively. This study included a control (isotype ADC), which was an ADC of an anti-mesothelin antibody and the dimer-linker of formula (IV).

Again, the single dose regimen was somewhat more efficacious, although exhibiting higher transient body weight loss. In the single dose study, tumor regression was observed in eight of eight mice at 0.3 μmol/kg and in four of eight mice at 0.1 μmol/kg. In the split dose study, tumor regression was observed in four of eight mice at 0.1 μmol/kg.

### Example 7

This example provides results of patient derived xenograft (PDX) studies.

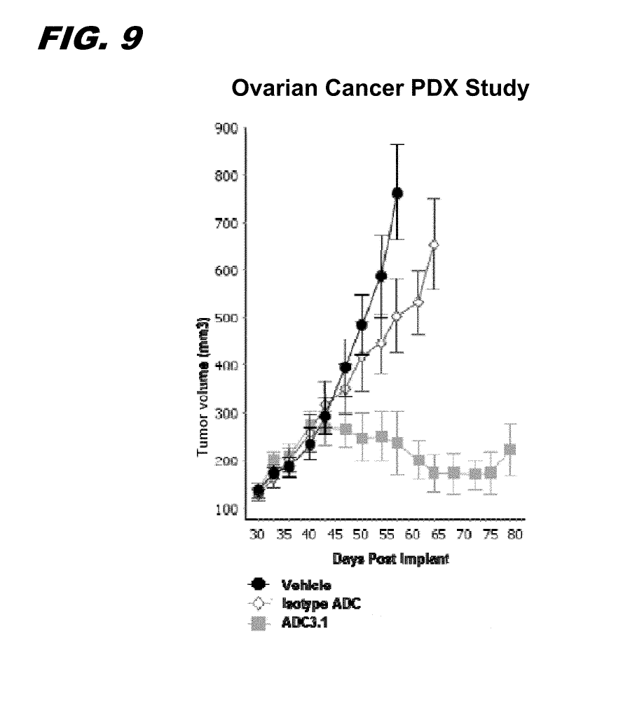

FIG. 9 shows the reduction in a patient-derived ovarian tumor volume upon treatment with ADC3.1. The dosing schedule was Q7D×6 (six weekly doses) and the dosage was 3 mg/kg. The control was a CD70 ADC carrying the same linker and drug moiety (formula (IV)), at a dosage of also 3 mg/kg but with a dosing schedule of three weekly doses (Q7D×3). First dosing was 30 days post-implantation.

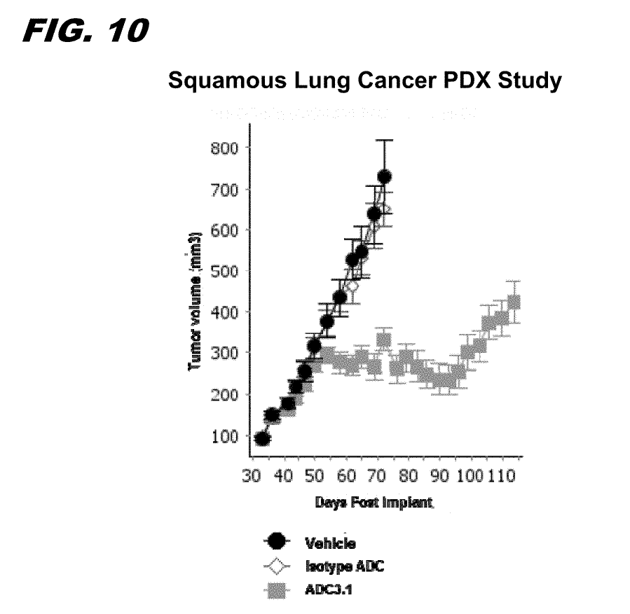

FIG. 10 shows the reduction in a patient derived squamous lung tumor volume upon treatment with ADC3.1. The dosing schedule was X7D×6 (six weekly doses) and the dosage was 3 mg/kg. The control was a mesothelin ADC carrying the same linker and drug moiety (formula (IV)), at a dosage of also 3 mg/kg but a dosing schedule was three weekly doses (Q7D×3). The first dosing was 30 days post-implantation.

### Example 8

This example describes a study seeking to identify variants of antibody GPC3.1, which might be better targeting agents for an ADC.

Anti-glypican-3 antibodies internalize with similar efficiencies into target cells such as cancerous cells that express high levels of glypican-3, regardless of whether they are low affinity binders (fast k_{off}, K_{D} ≈10 nM) or high affinity binders (slow k_{off}, K_{D}≦1 nM). Hypothetically, it is possible that an ADC of anti-glypican-3 antibody with relatively low affinity may exhibit reduced toxicity against normal cells, which have a lower expression level of glypican-3. Further, an ADC of an anti-glypican-3 antibody with relatively low affinity may distribute more facilely into distant tumor tissues. To evaluate this hypothesis, 83 variants of Antibody GPC3.1 were prepared, containing modifications in the variable region. Of these, the three most promising (designated antibodies A, B, and C) were selected for head-to-head comparisons against antibody GPC3.1.

Compared to Antibody GPC3.1, Antibody A has the same heavy chain CDR1 and light (kappa) chain CDR1 and CDR2 (SEQ ID NO;1, NO:4, and NO:5, respectively) but different heavy chain CDR2 and CDR3 (SEQ ID NO:17 and NO:18, respectively) and kappa chain CDR3 (SEQ ID NO:19). Also, Antibody A differs from Antibody GPC3.1 in certain heavy chain framework amino acids, as noted in its heavy chain variable region sequence (SEQ ID NO:23). Its kappa chain variable region sequence is provided in SEQ ID NO:24. Its heavy and kappa chain constant regions have the same sequence as those in Antibody GPC3.1 (SEQ ID NO:9 and NO:10, respectively).

Compared to Antibody GPC3.1, Antibody B has the same heavy chain CDR3 and light (kappa) chain CDR1 and CDR2 (SEQ ID NO:3, NO:4 and NO:5, respectively) but different heavy chain CDR1 and CDR2 (SEQ ID NO:20 and NO:21, respectively) and kappa chain CDR3 (SEQ ID NO:22). Also, Antibody B differs from Antibody GPC3.1 in certain heavy chain framework amino acids, as noted in its heavy chain variable region sequence (SEQ ID NO:25). Its kappa chain variable region sequence is provided in SEQ ID NO:26. Its heavy and kappa chain constant regions have the same sequence as those in Antibody GPC3.1 (SEQ ID NO:9 and NO:10, respectively).

Compared to Antibody GPC3.1, Antibody C has the same heavy chain CDR3 and light (kappa) chain CDR1 and CDR2 (SEQ ID NO:3, NO:4 and NO:5, respectively) but different heavy chain CDR1 and CDR2 (SEQ ID NO:20 and NO:17, respectively) and kappa chain CDR3 (SEQ ID NO:19). Also, Antibody C differs from Antibody GPC3.1 in certain heavy chain framework amino acids, as noted in its heavy chain variable region sequence (SEQ ID NO:27). Its kappa chain variable region sequence is provided in SEQ ID NO:28. Its heavy and kappa chain constant regions have the same sequence as those in Antibody GPC3.1 (SEQ ID NO:9 and NO:10, respectively).

The respective k_{off} and K_{D} values for antibodies A, B, C and GPC3.1 are shown in Table 2.

TABLE 2
Binding Affinity of Variant Antibodies to Glypican-3

| Antibody | K_{D} × 10⁻⁹ (M) | kₒₙ × 10⁴ (1/Ms) | k_{off} × 10⁻⁴ (1/s) |
|---|---|---|---|
| A | 2.7 | 110 | 29 |
| B | 2.2 | 170 | 36 |
| C | 0.9 | 170 | 16 |
| GPC3.1 | 28 | 150 | 415 |

Compared to antibody GPC3.1, variant antibodies A, B, and C exhibited between 10-and 30-fold improvement in K_{D} and k_{off} according to Biacore™ assays. They also exhibited faster clearance in mice.

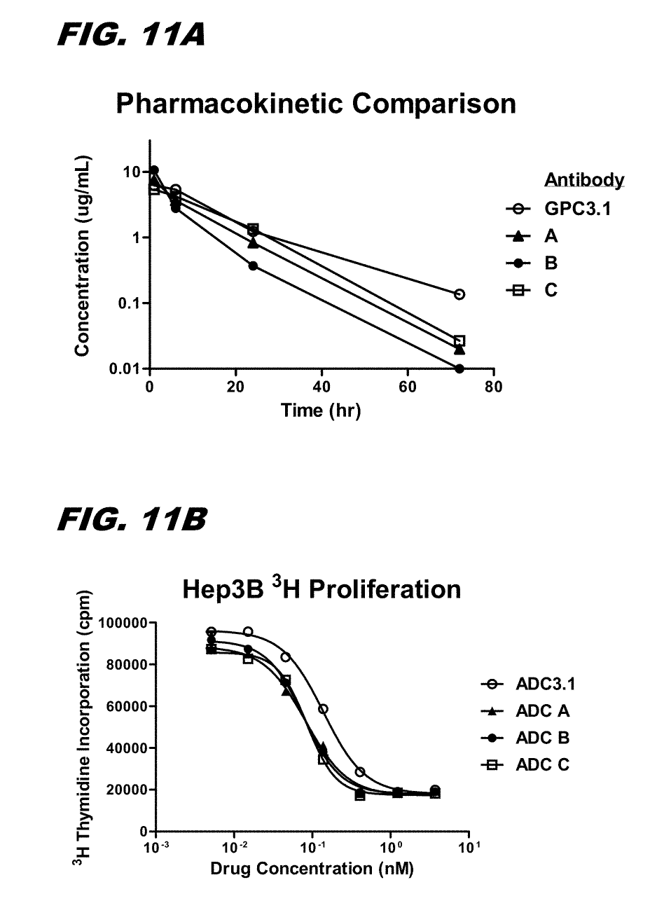

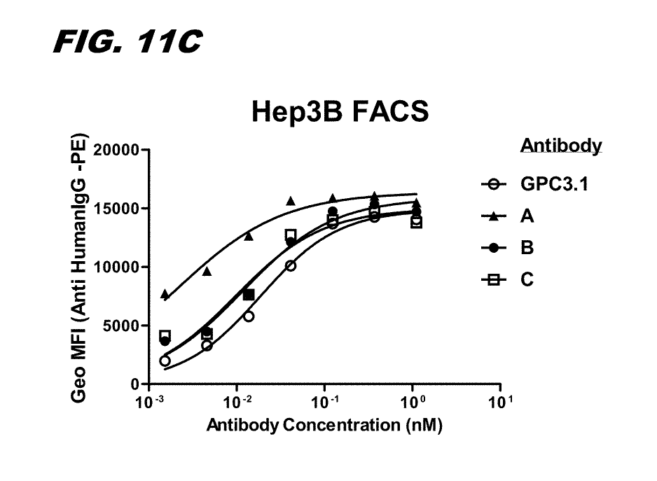

FIG. 11A is a comparison of the pharmacokinetic (PK) profiles in SCID mice at an intravenous dose of 0.5 mg/kg. The profiles were similar, although that of antibody GPC3.1 was slightly better.

FIG. 11B presents the results of a ³H thymidine incorporation in vitro assay using Hep3B hepatocellular carcinoma cells. According to these results, ADCs of antibodies A, B, and C with the drug-linker of formula (IV) (ADCs A, B, and C, respectively) were slightly more active than ADC3.1

FIG. 11C shows the results of a FACS (fluorescence activated cell sorting) study of the binding of the four ADCs of FIG. 11B. ADC A was slightly more active than the other three, which were similarly active.

Thus, in view of the foregoing in vitro results, ADCs A, B, and C were viewed as promising candidates for in vivo comparative studies against ADC3.1. The results of such studies are presented and discussed in Example 10 hereinbelow.

### Example 9

This example describes a different study with a similar objective, that is, to identify other anti-glypican-3 antibodies, which might be more efficacious as a targeting agent in an ADC than antibody GPC3.1.

Rather than modifying antibody GPC3.1, anti-glypican-3 antibodies were made de novo by immunizing HuMab® transgenic mice. Methods for raising of human antibodies by immunizing HuMab® transgenic mice are disclosed in Terrett et al., U.S. Pat. No. 8,680,247 B2 (2014), the disclosure of which is incorporated herein by reference. The binding properties of four antibodies so raised and antibody GPC3.1 are shown in Table 3 below.

TABLE 3
Binding Affinity of HuMab ® Antibodies to Glypican-3

| Antibody | K_{D} × 10⁻⁹ (M) | kₒₙ × 10⁴ (1/Ms) | k_{off} × 10⁻⁴ (1/s) |
|---|---|---|---|
| D | 3.9 | 19 | 74 |
| E | 0.5 | 15 | 0.7 |
| F | 13 | 27 | 36 |
| G | 133 | 2.1 | 28 |
| GPC3.1 | 28 | 147 | 415 |

Binning studies showed that antibody E bound to a different epitope than antibody GPC3.1 and was non-blocking vis-a-vis it. The epitope grouping of antibody D was not determined.

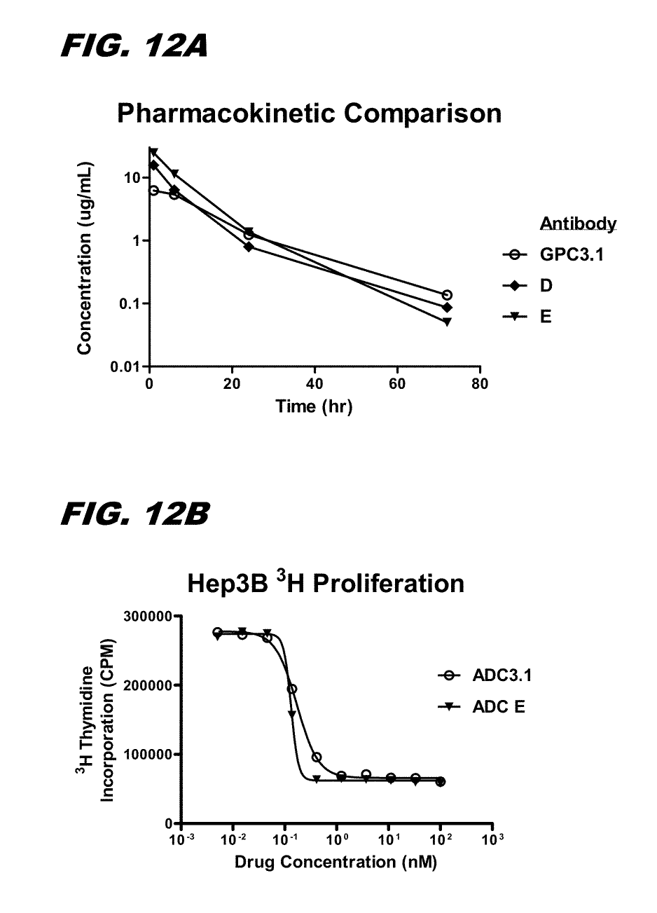

FIG. 12A shows that the PK profiles of antibodies GPC3.1, E, and E are similar. SCID mice were used, with a dose of 0.5 mg/kg administered intravenously. FIG. 12B shows that, in the in vitro Hep3B ³H thymidine incorporation assay, the activities of ADC3.1 and an ADC of antibody E with drug-linker (IV) (ADC E) are very close, with EC₅₀'s of 0.17 and 0.13 nM, respectively.

In view of the above results antibody E was selected for ADC comparative studies against ADC 3.1.

### Example 10

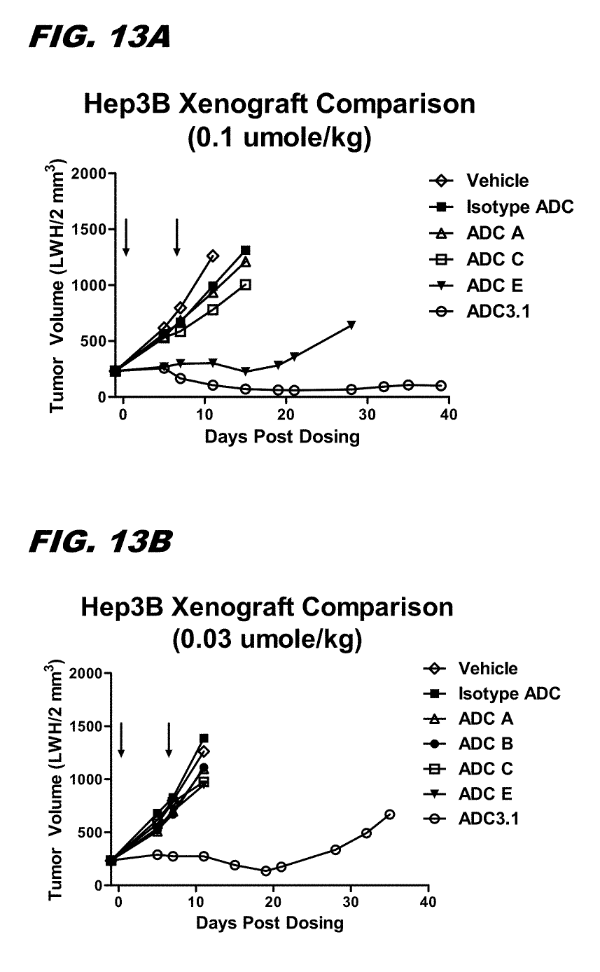

ADCs were prepared combining drug linker compound (IV) and antibodies A, B, C, and E, to produce ADCs respectively designated ADC A, ADC B, ADC C, and ADC E. In vivo xenograft studies were performed for these four ADCs alongside ADC 3.1, a vehicle (formulation buffer) control, and an ADC control (an ADC of an anti-CD70 antibody with drug-linker (IV)), using Hep3B cells. Dosing was 0.1 μmol/kg (5 mg/kg) in one case (FIG. 13A) and 0.03 μmol/kg (1.5 mg/kg) in the other case (FIG. 13B). The dosing frequency was Q7D×2. As can ben seen from these figures, none of ADCs A, B, C or E was as efficacious as antibody GPC3.1 in reducing the tumor volume. These results evidence the unpredictable nature of developing an efficacious ADC by extrapolating from in vitro results. The superior in vivo efficacy of ADC 3.1 in comparison with the other four ADCs was unexpected in view of the similarily in in vitro properties.

### Example 11

For comparative purposes, an ADC of anti-GPC3 antibody GPC3.1 and drug-linker compound (V) was prepared.

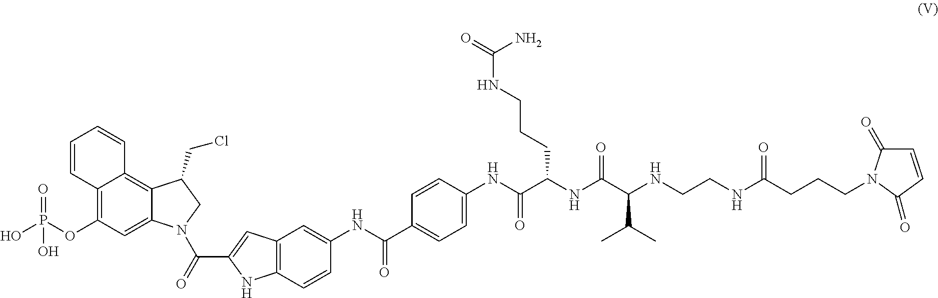 [SMILES: CC(C)[C@H](NCCNC(=O)CCCN1C(=O)C=CC1=O)C(=O)N[C@@H](CCCNC(N)=O)C(=O)Nc1ccc(C(=O)Nc2ccc3[nH]c(C(=O)N4C[C@@H](CCl)c5c4cc(OP(=O)(O)O)c4ccccc54)cc3c2)cc1]

The drug moiety in compound (V) belongs to the class of cytotoxins known as cyclopropabenzindoles (CBIs) and have been used in ADCs (Zhang et al. 2015).

When tested against Hep3B cancer cells by the ³H thymidine assay, the ADC of compound (V) had a potency (EC₅₀ 0.079 nM) that compared favorably against that of ADC3.1 (EC₅₀ 0.15 nM). However, the former's pharmacokinetic (PK) properties were not as desirable as that of ADC3.1.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

### REFERENCES

Full citations for the following references cited in abbreviated fashion by first author (or inventor) and date earlier in this specification are provided below. Each of these references is incorporated herein by reference for all purposes.

Cheng et al., U.S. Pat. No. 8,394,922 B2 (2013).

Cong et al., U.S. Pat. No. 8,980,824 B2 (2015).

Dubowchik et al., “Cathepsin B-Sensitive Dipeptide Prodrugs. 1. A Model Study of Structural Requirements for Efficient Release of Doxorubicin,” Biorg. Med. Chem. Lett. 1998, 8, 3341 [1998a].

Dubowchik et al., “Cathepsin B-Sensitive Dipeptide Prodrugs. 2. Models of Anticancer Drugs Paclitaxel (Taxol™), Mitomycin C, and Doxorubicin,” Bioorg. Med. Chem. Lett., 1998, 8, 3347 [1998b].

Dubowchik et al., “Cathepsin B-Labile Dipeptide Linkers for Lysosomal Release of Doxorubicin from Internalizing Immunoconjugates: Models Studies of Enzymatic Drug Release and Antigen-Specific in Vitro Anticancer Activity,” Bioconjugate Chem. 2002, 13, 855.

Ho et al., US 2014/0044714 A1 (2014).

Ho et al., US 2015/0147330 A1 (2015).

Jakubovic and Jothy, “Glypican-3: From the mutations of Simpson-Golabi-Behmel genetic syndrome to a tumor marker for hepatocellular carcinoma,” Exp. Mol. Path. 2007, 82, 184.

Luo et al., “Probing of C-Terminal Lysine Variation in a Recombinant Monoclonal Antibody Production Using Chinese Hamster Ovary Cells With Chemically Defined Media,” Biotechnol. Bioeng. 2012, 109 (5), 2306.

Nakatsura and Nishimura, “Usefulness of the Novel Oncofetal Antigen Glipcan-3 for Diagnosis of Hepatocellular Carcinoma and Melanoma,” Biodrugs 2005, 19 (2), 71.

Parikh and Hyman, “Hepatocellular Cancer: A Guide for the Internist,” Am. J. Med. 2007, 120 (3), 194.

Sasisekharan et al., “Roles of Heparan-Sulphate Glycosaminoglycans in Cancer,” Nature Rev. Cancer 2002, 2, 521.

Schrama et al.,“Antibody targeted drugs as cancer therapeutics,” Nature Rev. Drug Disc. 2006, 5, 147-159.

Smith et al., WO 2007/137170 A2 (2007).

Terrett et al., U.S. Pat. No. 8,680,247 B2 (2014).

Zhang et al., U.S. Pat. No. 8,852,599 B2 (2014).

Zhang et al., U.S. Pat. No. 9,186,416 B2 (2015).

### TABLE OF SEQUENCES

Table 4 below provides a short summary of the sequence listings filed together with this specification.

TABLE 4
Sequence Listing Summary

| SEQ ID NO: | SEQUENCE DESCRIPTION |
|---|---|
| 1 | GPC3.1 heavy chain CDR1 a.a. |
| 2 | GPC3.1 heavy chain CDR2 a.a. |
| 3 | GPC3.1 heavy chain CDR3 a.a. |
| 4 | GPC3.1 kappa chain CDR1 a.a. |
| 5 | GPC3.1 kappa CDR2 a.a. |
| 6 | GPC3.1 kappa CDR3 a.a. |
| 7 | GPC3.1 heavy chain variable region a.a. |
| 8 | GPC3.1 kappa chain variable region a.a. |
| 9 | GPC3.1 heavy chain constant region a.a. |
| 10 | GPC3.1 kappa chain constant region a.a. |
| 11 | GPC3.1 heavy chain a.a. |
| 12 | GPC3.1 kappa chain a.a. |
| 13 | GPC3.1 heavy chain with signal peptide n.t. |
| 14 | GPC3.1 heavy chain with signal peptide a.a. |
| 15 | GPC3.1 kappa chain with signal peptide n.t. |
| 16 | GPC3.1 kappa chain with signal peptide a.a. |
| 17 | Antibodies A and C heavy chain CDR2 a.a. |
| 18 | Antibody A heavy chain CDR3 a.a. |
| 19 | Antibodies A and C kappa chain CDR3 a.a. |
| 20 | Antibodies B and C heavy chain CDR1 a.a. |
| 21 | Antibody B heavy chain CDR2 a.a. |
| 22 | Antibody B kappa chain CDR3 a.a. |
| 23 | Antibody A heavy chain variable region a.a. |
| 24 | Antibody A kappa chain variable region a.a. |
| 25 | Antibody B heavy chain variable region a.a. |
| 26 | Antibody B kappa chain variable region a.a. |
| 27 | Antibody C heavy chain variable region a.a. |
| 28 | Antibody C kappa chain variable region a.a. |

What is claimed is:

1. An antibody-drug conjugate, having a structure represented by formula (I) 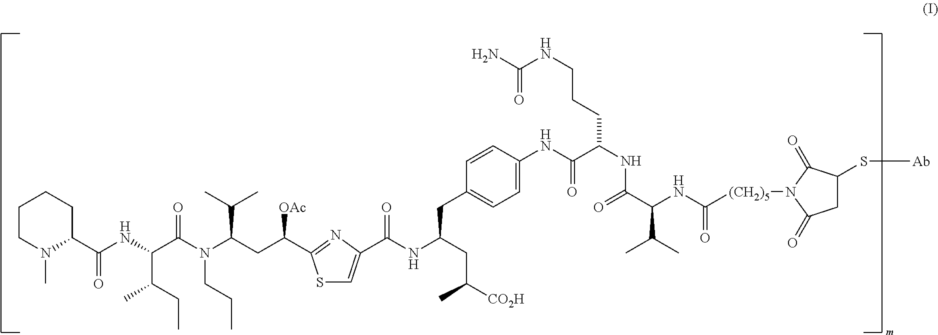
wherein
m is 1, 2, 3, or 4 and
Ab is an anti-glypican-3 antibody having
(a) a heavy chain variable region CDR1 comprising SEQ ID NO:1;
(b) a heavy chain variable region CDR2 comprising SEQ ID NO:2;
(c) a heavy chain variable region CDR3 comprising SEQ ID NO:3;
(d) a kappa light chain variable region CDR1 comprising SEQ ID NO:4;
(e) a kappa light chain variable region CDR2 comprising SEQ ID NO:5; and
(f) a kappa light chain variable region CDR3 comprising SEQ ID NO:6.

2. An antibody-drug conjugate according to claim 1, wherein the antibody Ab has a heavy chain variable region comprising SEQ ID NO:7 and a kappa light chain variable region comprising SEQ ID NO:8.

3. An antibody-drug conjugate according to claim 1, wherein the antibody Ab has a heavy chain constant region comprising SEQ ID NO:9 and a kappa light chain constant region comprising SEQ ID NO:10.

4. An antibody-drug conjugate according to claim 3, wherein the heavy chain constant region further comprises a C-terminal lysine.

5. An antibody-drug conjugate according to claim 1, wherein the antibody Ab has a heavy chain comprising SEQ ID NO:11 and a kappa light chain comprising SEQ ID NO:12.

6. An antibody-drug conjugate according to claim 5, wherein the heavy chain further comprises a C-terminal lysine.

7. A method of treating a cancer in a human subject suffering from such cancer, comprising administering to the human subject a therapeutically effective amount of an antibody-drug conjugate according to claim 1, 2, 3, 4, 5, or 6, where the cancer is hepatocellular carcinoma, ovarian, or lung cancer.

8. A method according to claim 7, wherein the antibody-drug conjugate is administered intravenously, at a dose of between 0.1 and 20 mg/kg.

9. A pharmaceutical formulation comprising an antibody-drug conjugate according to claim 1, 2, 3, 4, 5, or 6, and a pharmaceutically acceptable excipient.
